(19) United States
(12) Patent Application Publication
(10) Pub. No.: US 2026/0337520 A1
(43) Pub. Date: Oct. 29, 2026

(54) SOPHOROLIPID SURFACTANTS WITH SURFACE-ACTIVE COUNTERCATIONS

(71) Applicant: Henkel AG & Co. KGaA, Düsseldorf (DE)

(72) Inventors: Janine Birnbach, Leverkusen (DE); Peter Schmiedel, Düsseldorf (DE); Cara Kohler, Düsseldorf (DE)

(21) Appl. No.: 19/119,273

(22) PCT Filed: Aug. 14, 2023
(86) PCT No.: PCT/EP2023/072365

§ 371 Date: Apr. 8, 2025

(30) Foreign Application Priority Data
Oct. 14, 2022 (DE) 10 2022 210 849.3

(51) Int. Cl. C11D 1/65; A61K 8/60; A61Q 5/02; C07H 15/10; C11D 1/36; C11D 1/62

(52) U.S. Cl. CPC C11D 1/65; A61K 8/60; A61Q 5/02; C07H 15/10; C11D 1/36; C11D 1/62

(57) ABSTRACT

The invention was to provide washing- and cleaning-active ingredients that enable the production of visually appealing, concentrated, free-flowing washing, cleaning and hair treatment product preparation and are producible in a simple and efficient manner, have good storability and have the particular feature of good washing and cleaning outcomes. This was achieved by compounds of the general formula (I) S—CO₂—N+(R¹R²R³R⁴) (I) in which S—CO₂— is a sophorolipid carboxylate anion and R¹ is selected from R² and H, R² is selected from C₁ to C₄ alkyl radicals and C₂ to C₄ hydroxyalkyl radicals, R³ is selected from R⁴ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, where R⁵ is selected from the C₂ to C₄ alkylene radicals and R⁶ is selected from the C₁₀ to C₁₈ alkyl radicals, and the alkyl moieties in each of R¹, R², R³, R⁴ and R⁶ may be linear or branched.

The present invention relates to a wash-active compound. Furthermore, the application relates to washing, cleaning and hair treatment agents which contain this wash-active compound, as well as washing, cleaning and hair treatment methods using the wash-active compound.

Continuously changing requirements are placed on the ways in which washing, cleaning and hair treatment agents are formulated and offered. A trend relevant to the production of such agents is the concentration thereof. In addition to higher consumer acceptance due to simplified handling, the background of this development is, in particular, sustainability aspects, for example in relation to transport volumes and costs and the quantity of packaging materials used.

The concentration of modern washing, cleaning and hair treatment agents, in particular modern liquid washing agents, generally influences their optical and rheological properties, affects the storage stability of these agents, and can influence their cleaning performance, in particular when the high concentration of the active substances leads to cases of incompatibility.

In addition, the concentration of liquid agents generally involves the use of organic solvents. These solvents improve the physical stability of the washing, cleaning and hair treatment agents, but are often characterized by only low washing or cleaning activity, as a result of which they should be added in the smallest possible quantities, not least for sustainability reasons. However, the use of aqueous or aqueous-organic solvent systems in the production of concentrated liquid agents cannot yet be avoided with conventional wash-active or cleaning-active agents.

Washing agents are known from the patent application EP 0 499 434 A1 that contain a surfactant forming a micellar phase and another surfactant forming a lamellar phase, wherein at least one of these two surfactants must be a glycolipid biosurfactant, in particular a rhamnolipid, glucose lipid, sophorose lipid, trehalose lipid and/or cellobiose lipid. European patent application EP 1 445 302 A1 relates to washing agents containing at least one glycolipid biosurfactant, in particular sophorolipid and/or rhamnolipid, and at least one non-glycolipid surfactant, these surfactants being in a micellar phase. Liquid aqueous washing agents are known from European patent application EP 3 686 265 A1 that contain anionic surfactant of the sulfate or sulfonate type, soap, nonionic surfactant, enzyme and sophorolipid. European patent application EP 3 290 501 A1 relates to agents containing alkoxylated fatty acid amide and a glycolipid biosurfactant, which can be a sophorolipid.

The object addressed by the present invention was that of providing wash-active and cleaning-active active ingredients that allow for the production of visually appealing, concentrated and flowable preparations of washing, cleaning and hair treatment agent. The active ingredients should be producible in a simple and efficient manner, have good storability, and be characterized in particular by good washing and cleaning results.

This object was achieved by a new class of surfactant active ingredients.

The invention first relates to a compound of general formula (I)

S—CO₂⁻N⁺(R¹R²R³R⁴) (I)
in which
S—CO₂⁻ is a sophorolipid carboxylate anion and
R¹ is selected from R² and H, R² is selected from C₁ to C₄ alkyl radicals and C₂ to C₄ hydroxyalkyl radicals, R³ is selected from R¹ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, wherein R⁵ is selected from the C₂ to C₄ alkylene radicals and R⁶ is selected from the C₁₀ to C₁₈ alkyl radicals, and in each of R¹, R², R³, R⁴ and R⁶, the alkyl moieties can be linear or branched-chain.

Surprisingly, it has been found that the neutralization of sophorolipids with tertiary amines or the exchange of the metal cations of sophorolipid metal salts for quaternary ammonium cations leads to surface-active compounds whose washing performance is superior to that of sophorolipids or sophorolipid metal salts. The surfactants obtainable in this way are suitable for the production of washing, cleaning and hair treatment agents, for example for the production of solvent-free liquid agents having active ingredient contents of up to 100 wt. %. In contrast to some other anionic surfactant-cationic surfactant combinations, the substances essential to the invention do not form poorly soluble or insoluble precipitates.

Sophorolipids consist of a sophorose radical in which two glucose molecules are connected to one another by a ß-1,2′-glycosidic bond, to which glucose molecules a hydroxyl group of a hydroxycarboxylic acid is connected by means of a ß-glycosidic bond. The hydroxycarboxylic acid can bear the hydroxyl group on the terminal C atom or on a C atom located within the carbon chain and can be saturated or unsaturated. Examples of sophorolipids are:

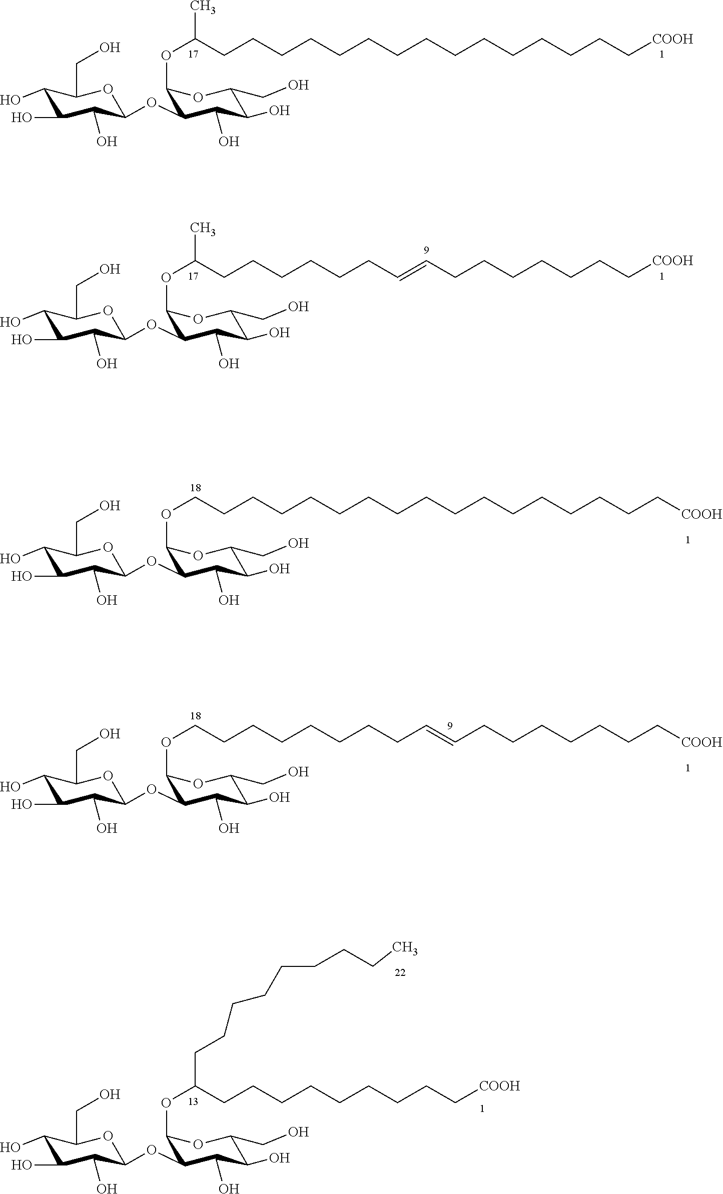

They can be in the open form shown above by way of example, or in the form of monomeric or dimeric lactones, for instance:

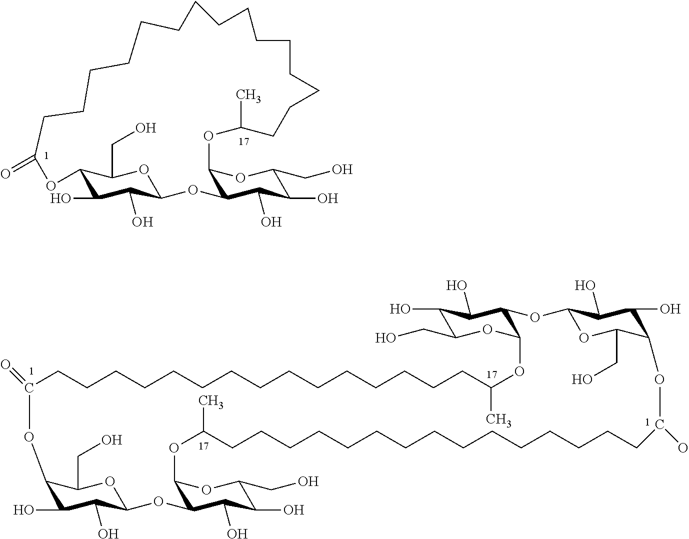

The open form and the lactone form can also exist alongside one another, and can both be converted into one another by intramolecular esterification or hydrolysis. In the compounds of formula (I), the sophorolipids are present in the open form. The hydroxyl groups located on the sophorose radicals of the sophorolipids can also be esterified, either completely or partially, using carboxylic acids, with acetic acid being an example of a carboxylic acid, and the primary hydroxyl groups being the prime esterification points. Sophorolipids are formed, for example, by yeasts, such as Candida albicans, Candida apicola, Candida floricola, Candida kuoi, Candida riodocensis, Candida stellata, Candida tropicalis, Candida parapsilosis, Rhodotorula bajevae, Rhodotorula bogoriensis, Wickerhamiella domercqiae, Wickerhamomyces anomalus and Starmerella bombicola, and can be obtained from the fermentation of such microorganisms.

In one embodiment of the invention, the cations N⁺(R¹R²R³R⁴) of the compounds of general formula (I) preferably have two to three, in particular three, of the four radicals with a carbon chain length of at most 4, in particular from 1 to 2. The remaining radical or the other two radicals have a longer carbon chain.

The compounds of general formula (I) have relatively low surface tension, which is normally in the range of 30 mN/m to 40 mN/m, and a relatively low critical micelle concentration, which is normally in the range of 0.001 mmol/1 to 0.02 mmol/l. They are highly soluble in water. A further advantage of the compounds of general formula (I) is that they produce a stable foam in aqueous solution, which can also be produced for at least 2 minutes in an amount that continuously increases over time.

The invention further relates to mixtures of compounds of general formula (I) with sophorolipids which do not have an ammonium countercation essential to the invention, but are in the form of the free acid or its alkali salts, or which are in lactone form, and to mixtures of compounds of general formula (I) with salts which contain cations corresponding to N⁺(R¹R²R³R⁴) in formula (I) but have a different counteranion, for example a halide. It is possible, although less preferred, that the mixtures contain both sophorolipids without a countercation as defined in general formula (I) and ammonium cations without a sophorolipid carboxylate anion as defined in formula (I). The mixtures mentioned in this paragraph contain a) the compound of general formula (I) and b) the sophorolipid not defined in general formula (I) and/or the cation not defined in general formula (I), preferably in weight ratios of a) to b) in ranges from 1:100 to 100:1, in particular from 100:1 to 50:50.

As stated at the outset, the compounds of general formula (I) are particularly suitable as components of washing, cleaning or hair treatment agents, in particular liquid washing, cleaning or hair treatment agents, due to their physical properties. Because of their high washing or cleaning power, the amount of surfactant in such agents can be reduced while maintaining the same performance, or a smaller amount of the agent is needed to achieve a good washing or cleaning result. The invention therefore further relates to a cleaning or hair treatment agent containing a compound of general formula (I) or a mixture as defined in the preceding paragraph.

A washing agent is a composition which is suitable for cleaning or caring for textiles and contains at least one textile-cleaning or textile-care active substance. Examples of textile-cleaning active substances are surfactants, in particular nonionic and anionic surfactants, and builders, such as phosphonates or water-softening polymers. Examples of textile-care active ingredients are fragrances, in particular encapsulated fragrances, textile softeners, in particular cationic surfactants and cationic polymers, and dyes, in particular textile dyeing agents.

The invention also relates to a method for washing textiles, during which method a compound of general formula (I) or a washing agent containing a compound of general formula (I) or a mixture according to the invention as defined above is introduced into a washing liquor which contains a textile in need of cleaning or into which a textile in need of cleaning is introduced.

A cleaning agent is a composition which is suitable for cleaning and caring for hard surfaces and contains at least one surface-cleaning or surface-care active substance. Examples of surface-cleaning active substances are surfactants, in particular nonionic and anionic surfactants, and builders, such as phosphonates or water-softening polymers. Examples of surface-care active substances are corrosion inhibitors, for instance zinc salts, and hydrophobizing agents, in particular polymeric hydrophobizing agents.

The method also relates to a method for cleaning hard surfaces, during which method a compound of general formula (I) or a mixture according to the invention as defined above or a cleaning agent containing a compound of general formula (I) or a mixture according to the invention as defined above is introduced into a cleaning liquor which is brought into contact with the hard surface to be cleaned.

A hair treatment agent is a composition which is suitable for cleaning and caring for hair, especially human hair. Hair treatment agents are understood to mean in particular hair cleaning agents, such as shampoos, hair care agents, such as hair masks, conditioners or hair care sprays as well as hair styling agents, such as hair gels, hair sprays or hair waxes. The hair treatment agent is preferably a shampoo or a hair care agent.

The invention also relates to a method for treating hair, during which method a compound of general formula (I) or a mixture or a hair treatment agent according to the invention as defined above containing a compound of general formula (I) or a mixture according to the invention as defined above is applied in particular to wet hair. If the hair treatment agent is a shampoo, a conditioner or a hair mask, it is preferably left to act on the hair for at least 5 seconds, preferably for a period of 5 seconds to 5 minutes, and then rinsed off with water.

A washing, cleaning or hair treatment agent according to the invention preferably contains 0.1 wt. % to 40 wt. %, in particular 0.2 wt. % to 30 wt. %, and particularly preferably 0.5 wt. % to 15 wt. %, of the compound of general formula (I) or the mixture according to the invention defined above. In addition to the compound of general formula (I) or the aforementioned mixture, it may contain other conventional components of such agents.

When producing washing, cleaning and hair treatment agents, the compound of general formula (I) can be produced by combining sophorolipid and cationic surfactant which contains a cation N⁺(R¹R²R³R⁴) as defined for the compound of general formula (I).

In the context of the present invention, a washing or cleaning agent preferably contains, in addition to the compound of general formula (I) or the above-defined mixture according to the invention, one or more substances from the group of nonionic surfactants, anionic surfactants, builders, bleaching agents, bleach activators, enzymes, electrolytes, pH adjusters, perfumes, perfume carriers, fluorescent agents, dyes, hydrotropes, foam inhibitors, anti-redeposition agents, graying inhibitors, shrinkage inhibitors, anti-crease agents, dye transfer inhibitors, antimicrobial active ingredients, non-aqueous solvents, germicides, fungicides, antioxidants, preservatives, corrosion inhibitors, antistatic agents, bittering agents, ironing aids, repellents and impregnating agents, skin care active ingredients, anti-swelling and anti-slip agents, plasticizing components and UV absorbers.

In the context of the present invention, a hair treatment agent preferably contains, in addition to the compound of general formula (I) or the above-defined mixture according to the invention, one or more substances from the group of non-ionic surfactants, amphoteric/zwitterionic surfactants, non-ionic polymers, anionic polymers, amino acids, oligopeptides, vitamins, provitamins, vitamin precursors, betaines, bioquinones, purine (derivatives), taurine (derivatives), L-carnitine (salts), panthenol, pantothenic acid, 2-furanones, 2-furanone derivatives, ectoin, allantoin, plant extracts, ester oils, UV light protection filters, structuring agents, thickeners, electrolytes, pH adjusters, swelling agents, dyes, anti-dandruff active ingredients, complexing agents, opacifiers, pearlescent agents, pigments, stabilizers, foaming agents, antioxidants, perfume oils, and/or preservatives.

A washing, cleaning or hair treatment agent according to the invention can preferably contain up to 30 wt. % of additional surfactant, wherein any surfactants additionally present are preferably also obtainable from renewable raw materials. In one particular embodiment of the invention, the agent contains up to 20 wt. %, in particular 0.5 wt. % to 15 wt. %, of nonionic surfactant.

Suitable nonionic surfactants include alkoxylated fatty acid alkyl esters, fatty acid amides, alkoxylated fatty acid amides, polyhydroxy fatty acid amides, alkylphenol polyglycol ethers, amine oxides, alkyl polyglycosides, and mixtures thereof. In addition, alkyl glycosides of the general formula R⁵O(G), can also be used as further nonionic surfactants, in which formula R⁵ corresponds to a primary straight-chain or methyl-branched, in particular methyl-branched at position 2, aliphatic radical having 8 to 22, preferably 12 to 18, C atoms, and G is the symbol representing a glycose unit having 5 or 6 C atoms, preferably glucose. The degree of oligomerization x, which indicates the distribution of monoglycosides and oligoglycosides, is any number between 1 and 10; x is preferably between 1.2 and 1.4.

Another class of nonionic surfactants which are used either as the sole nonionic surfactant or in combination with other additional nonionic surfactants, are alkoxylated, preferably ethoxylated or ethoxylated and propoxylated, fatty acid alkyl esters, preferably having 1 to 4 carbon atoms in the alkyl chain.

Nonionic surfactants of the aminoxide type, for example N-coco alkyl-N,N-dimethylamine oxide and N-tallow alkyl-N,N-dihydroxyethyl amine oxide, and fatty acid alkanolamide type may also be used.

Other optionally suitable surfactants are polyhydroxy fatty acid amides of the formula,

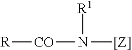

in which R represents an aliphatic acyl radical having 6 to 22 carbon atoms, R¹ represents hydrogen, an alkyl or hydroxyalkyl radical having 1 to 4 carbon atoms and [Z] represents a linear or branched polyhydroxyalkyl radical having 3 to 10 carbon atoms and 3 to 10 hydroxyl groups. The polyhydroxy fatty acid amides are known substances that can usually be obtained by the reductive amination of a reducing sugar with ammonia, an alkyl amine or an alkanol amine, and subsequent acylation with a fatty acid, a fatty acid alkyl ester or a fatty acid chloride. The group of polyhydroxy fatty acid amides also includes compounds of the formula

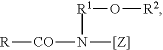

where R denotes a linear or branched alkyl or alkenyl radical having 7 to 12 carbon atoms, R¹ denotes a linear, branched or cyclic alkyl radical or an aryl radical having 2 to 8 carbon atoms, and R² denotes a linear, branched or cyclic alkyl radical or an aryl radical or an oxy alkyl radical having 1 to 8 carbon atoms, wherein Cia alkyl or phenyl radicals are preferred, and [Z] denotes a linear polyhydroxy alkyl radical, the alkyl chain of which is substituted with at least two hydroxyl groups, or alkoxylated, preferably ethoxylated or propoxylated derivatives of this radical. [Z] is preferably obtained by the reductive amination of a reduced sugar, for example glucose, fructose, maltose, lactose, galactose, mannose or xylose. The N-alkoxy- or N-aryloxy-substituted compounds can be converted into the desired polyhydroxy fatty acid amides by reaction with fatty acid methyl esters in the presence of an alkoxide as a catalyst.

A washing or cleaning agent according to the invention preferably contains at least one water-soluble and/or water-insoluble, organic and/or inorganic builder. The water-soluble organic builder substances include polycarboxylic acids, in particular citric acid and saccharic acids, monomeric and polymeric aminopolycarboxylic acids, in particular glycinediacetic acid, methylglycinediacetic acid, nitrilotriacetic acid, iminodisuccinates, such as ethylenediamine-N,N′-disuccinic acid, and hydroxyiminodisuccinates, ethylenediaminetetraacetic acid and polyaspartic acid, polyphosphonic acids, in particular aminotris(methylenephosphonic acid), ethylenediamine tetrakis(methylenephosphonic acid), lysine tetra(methylenephosphonic acid) and 1-hydroxyethane-1,1-diphosphonic acid, polymeric hydroxy compounds, such as dextrin, and polymeric (poly)carboxylic acids, polycarboxylates which can be obtained in particular by oxidizing polysaccharides, polymeric acrylic acids, methacrylic acids, maleic acids, and mixed polymers thereof, which may also contain small proportions of polymerizable substances without carboxylic acid functionality that are incorporated by polymerization. The relative average molecular mass of the homopolymers of unsaturated carboxylic acids is generally between 5,000 g/mol and 200,000 g/mol, that of the copolymers between 2,000 g/mol and 200,000 g/mol, preferably 50,000 g/mol to 120,000 g/mol, in each case based on free acid. A particularly preferred acrylic acid-maleic acid copolymer has a relative average molecular mass of from 50,000 to 100,000. Suitable, albeit less preferred compounds of this class are copolymers of acrylic acid or methacrylic acid with vinyl ethers, such as vinyl methyl ethers, vinyl esters, ethylene, propylene, and styrene, in which the proportion of the acid is at least 50 wt. %. It is also possible to use, as water-soluble organic builder substances, terpolymers which contain two unsaturated acids and/or the salts thereof as monomers and vinyl alcohol and/or a vinyl alcohol derivative or a carbohydrate as the third monomer. The first acid monomer or the salt thereof is derived from a monoethylenically unsaturated C₃-C₈ carboxylic acid and preferably from a C₃-C₄ monocarboxylic acid, in particular from (meth)acrylic acid. The second acid monomer or the salt thereof can be a derivative of a C₄-C₈ dicarboxylic acid, maleic acid being particularly preferred. In this case, the third monomeric unit is formed from vinyl alcohol and/or preferably an esterified vinyl alcohol. In particular, vinyl alcohol derivatives are preferred, which are an ester of short-chain carboxylic acids, for example C₁-C₄ carboxylic acids, with vinyl alcohol. Preferred polymers contain 60 wt. % to 95 wt. %, in particular 70 wt. % to 90 wt. %, (meth)acrylic acid or (meth)acrylate, particularly preferably acrylic acid or acrylate, and maleic acid or maleate, and 5 wt. % to 40 wt. %, preferably 10 wt. % to 30 wt. %, vinyl alcohol and/or vinyl acetate. Very particularly preferred are polymers in which the weight ratio of (meth)acrylic acid or (meth)acrylate to maleic acid or maleate is between 1:1 and 4:1, preferably between 2:1 and 3:1, and in particular between 2:1 and 2.5:1. Both the amounts and the weight ratios are based on the acids. The second acid monomer or the salt thereof can also be a derivative of an allylsulfonic acid which is substituted in the 2-position by an alkyl radical, preferably a C₁-C₄ alkyl radical, or an aromatic radical which is preferably derived from benzene or benzene derivatives. Preferred terpolymers contain from 40 wt. % to 60 wt. %, in particular from 45 to 55 wt. %, (meth)acrylic acid or (meth)acrylate, particularly preferably acrylic acid or acrylate, from 10 wt. % to 30 wt. %, preferably 15 wt. % to 25 wt. %, methallylsulfonic acid or methallyl sulfonate and 15 wt. % to 40 wt. %, preferably 20 wt. % to 40 wt. %, of a carbohydrate as the third monomer. This carbohydrate may be, for example, a mono-, di-, oligo- or polysaccharide, mono-, di- or oligosaccharides being preferred. Sucrose is particularly preferred. The use of the third monomer presumably incorporates predetermined breaking points into the polymer which are responsible for the good biodegradability of the polymer. These terpolymers generally have a relative average molecular mass between 1,000 g/mol and 200,000 g/mol, preferably between 200 g/mol and 50,000 g/mol. Further preferred copolymers are those which have acrolein and acrylic acid/acrylic acid salts or vinyl acetate as monomers. The organic builder substances may, in particular for the preparation of liquid agents, be used in the form of aqueous solutions, preferably in the form of 30 to 50 wt. % aqueous solutions. All of said acids are generally used in the form of their water-soluble salts, in particular their alkali salts.

Organic builder substances of this kind can, if desired, be contained in amounts of up to 40 wt. %, in particular up to 25 wt. %, and preferably from 1 wt. % to 8 wt. %. Amounts in the upper half of the stated ranges are preferably used in pasty or liquid washing or cleaning agents, in particular containing water.

In particular polyphosphates, preferably sodium triphosphate, are suitable as water-soluble inorganic builder materials. In particular, crystalline or amorphous, water-dispersible alkali aluminosilicates are used as water-insoluble inorganic builder materials in amounts not exceeding 25 wt. %, preferably from 3 wt. % to 20 wt. %, and in particular in amounts of from 5 wt. % to 15 wt. %. Of these, the crystalline sodium aluminosilicates of washing agent quality, particularly zeolite A, zeolite P, and zeolite MAP, and optionally zeolite X, are preferred. Amounts close to the stated upper limit are preferably used in solid, particulate agents. Suitable aluminosilicates in particular do not have any particles having a particle size greater than 30 μm and preferably consist, at least 80 wt. % thereof, of particles having a size smaller than 10 μm. The calcium binding capacity of said aluminosilicates is generally in the range of from 100 to 200 mg CaO per gram.

In addition or as an alternative to said water-insoluble aluminosilicate and alkali carbonate, further water-soluble inorganic builder materials may be contained. These include, in addition to the polyphosphates, such as sodium triphosphate, in particular the water-soluble crystalline and/or amorphous alkali silicate builders. Water-soluble inorganic builder materials of this kind are contained in the agents, preferably in amounts of from 1 wt. % to 20 wt. %, in particular from 5 wt. % to 15 wt. %. The alkali silicates that can be used as builder materials preferably have a molar ratio of alkali oxide to SiO₂ of less than 0.95, in particular of from 1:1.1 to 1:12, and may be present in amorphous or crystalline form. Preferred alkali silicates are sodium silicates, in particular amorphous sodium silicates, having a molar ratio of Na₂O:SiO₂ of from 1:2 to 1:2.8. Crystalline phyllosilicates of the general formula Na₂SiₓO₂ₓ₊₁ y H₂O, where x, referred to as the module, is a number from 1.9 to 4, y is a number from 0 to 20, and preferred values for x are 2, 3 or 4, are preferably used as crystalline silicates, which may be present on their own or in a mixture with amorphous silicates. Preferred crystalline phyllosilicates are those in which x assumes the values 2 or 3 in the mentioned general formula. In particular, both ß- and δ-sodium disilicates (Na₂Si₂O₅ y H₂O) are preferred. Practically water-free crystalline alkali silicates of the above general formula, in which x means a number from 1.9 to 2.1, and which are prepared from amorphous alkali silicates, may also be used in the agents. In another preferred embodiment, a crystalline sodium sheet silicate having a modulus of 2 to 3 of the like that can be produced from sand and soda is used. Sodium silicates having a module in the range of 1.9 to 3.5 are used in a further embodiment. In a preferred embodiment of such agents, a granular compound of alkali silicate and alkali carbonate is used, as is commercially available, for example, under the name Nabion® 15.

Peroxidic bleaching agents suitable in washing and cleaning agents may be in particular organic peracids or peracid salts of organic acids, such as phthalimidopercaproic acid, perbenzoic acid, monoperoxyphthalic acid, and diperdodecane diacid and salts thereof, such as magnesium monoperoxyphthalate, diacyl peroxides, hydrogen peroxide and inorganic salts which release hydrogen peroxide under the use conditions, such as alkali perborate, alkali percarbonate and/or alkali persilicate, and hydrogen peroxide inclusion compounds, such as H₂O₂ urea adducts, and mixtures thereof. Hydrogen peroxide can also be produced by means of an enzymatic system, i.e., an oxidase and the substrate thereof. If solid peroxygen compounds are intended to be used, these may be used in the form of powders or granules, which may also be coated in a manner known in principle. Particularly preferably, alkali percarbonate, alkali perborate monohydrate or hydrogen peroxide is used. A washing agent which can be used in the context of the invention contains peroxidic bleaching agent in amounts of preferably up to 60 wt. %, in particular of 5 wt. % to 50 wt. %, and particularly preferably of 15 wt. % to 30 wt. % or, alternatively, of 2.5 wt. % to 20 wt. %, hydrogen peroxide being the most preferred peroxidic bleaching agent in liquid agents and sodium percarbonate being most preferred in solid agents. Peroxidic bleaching agent particles preferably have a particle size in the range of from 10 μm to 5000 μm, in particular from 50 μm to 1000 μm, and/or a density from 0.85 g/cm³ to 4.9 g/cm³, in particular from 0.91 g/cm³ to 2.7 g/cm³.

In particular compounds which produce, under perhydrolysis conditions, optionally substituted perbenzoic acid and/or aliphatic peroxycarboxylic acids having 1 to 12 C atoms, in particular 2 to 4 C atoms, alone or in mixtures, are used as a bleach-activating compound that produces peroxycarboxylic acid under perhydrolysis conditions. Bleach activators that have O- and/or N-acyl radicals in particular of the stated number of C atoms and/or optionally substituted benzoyl radicals are suitable. Preferred are polyacylated alkylenediamines, in particular tetraacetylethylenediamine (TAED), acylated glycolurils, in particular tetraacetylglycoluril (TAGU), acylated triazine derivatives, in particular 1,5-diacetyl-2,4-dioxohexahydro-1,3,5-triazine (DADHT), N-acylimides, in particular N-nonanoylsuccinimide (NOSI), acylated phenolsulfonates or carboxylates or the sulfonic or carboxylic acids thereof, in particular nonanoyl- or isononanoyl- or lauroyloxybenzenesulfonate (NOBS or iso-NOBS or LOBS) or decanoyloxybenzoate (DOBA), the formal carbonic acid ester derivatives thereof, such as 4-(2-decanoyloxyethoxycarbonyloxy)-benzenesulfonate (DECOBS), acylated polyhydric alcohols, in particular triacetin, ethylene glycol diacetate and 2,5-di-acetoxy-2,5-dihydrodrofuran and acetylated sorbitol and mannitol and mixtures thereof (SORMAN), acylated sugar derivatives, in particular pentaacetylglucose (PAG), pentaacetylfructose, tetraacetylxylose and octaacetyl lactose, acetylated, optionally N-alkylated glucamine and gluconolactone, and/or N-acylated lactams, for example N-benzoyl-caprolactam.

In addition to, or instead of, the compounds which form peroxycarboxylic acids under perhydrolysis conditions, other bleach activating compounds, such as nitriles, from which perimidic acids form under perhydrolysis conditions, may be present. These include in particular aminoacetonitrile derivatives having a quaternized nitrogen atom according to the formula

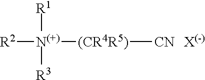

in which R¹ represents —H, —CH₃, a C₂₋₂₄ alkyl or alkenyl radical, a substituted C₁₋₂₄ alkyl or C₂₋₂₄ alkenyl radical having at least one substituent from the group —Cl, —Br, —OH, —NH₂, —CN and —N⁽⁺⁾—CH₂—CN, an alkyl or alkenylaryl radical having a C₁₋₂₄ alkyl radical, or representing a substituted alkyl or alkenylaryl radical having at least one, preferably two, optionally substituted C₁₋₂₄ alkyl radical(s) and optionally further substituents on the aromatic ring, R² and R³ are selected, independently of one another, from —CH₂—CN, —CH₃, —CH₂—CH₃, CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂CH₂—O)ₙH, where n=1, 2, 3, 4, 5 or 6, R⁴ and R⁵ have, independently of one another, a meaning specified above for R¹, R² or R³, where at least 2 of the radicals mentioned, in particular R² and R³, including the nitrogen atom and optionally other heteroatoms, can be linked to one another in a ring-closing manner and then preferably form a morpholino ring, and X is a charge-balancing anion, preferably selected from benzenesulfonate, toluenesulfonate, cumenesulfonate, the C₉₋₁₅ alkylbenzene sulfonates, the C₁₋₂₀ alkyl sulfates, the C₈₋₂₂ carboxylic acid methyl ester sulfonates, sulfate, hydrogen sulfate, and mixtures thereof, may be used. Bleach activators forming peroxycarboxylic acids or perimidic acids under perhydrolysis conditions are preferably present in agents according to the invention in amounts of up to 25 wt. %, in particular 0.1 wt. % to 10 wt. %. Bleach activator particles preferably have a particle size in the range of from 10 μm to 5000 μm, in particular from 50 μm to 1000 μm, and/or a density from 0.85 g/cm³ to 4.9 g/cm³, in particular from 0.91 g/cm³ to 2.7 g/cm³.

The presence of bleach-catalyzing transition metal complexes, in addition to or instead of said bleach activators, is possible. These are preferably selected from the cobalt, iron, copper, titanium, vanadium, manganese and ruthenium complexes. Suitable ligands in such transition metal complexes are both inorganic and organic compounds, which include, in addition to carboxylates, in particular compounds having primary, secondary and/or tertiary amine and/or alcohol functions, such as pyridine, pyridazine, pyrimidine, pyrazine, imidazole, pyrazole, triazole, 2,2′-bispyridylamine, tris-(2-pyridylmethyl)amine, 1,4,7-triazacyclononane, 1,4,7-trimethyl-1,4,7-triazacyclononane, 1,5,9-trimethyl-1,5,9-triazacyclododecane, (bis-((1-methylimidazole-2-yl)-methyl))-(2-pyridylmethyl)-amine, N,N′-(bis-(1-methylimidazole-2-yl)-methyl)-ethylenediamine, N-bis-(2-benzimidazolylmethyl) aminoethanol, 2,6-bis-(bis-(2-benzimidazolylmethyl)aminomethyl)-4-methylphenol, N,N,N′,N′-tetrakis-(2-benzimidazolylmethyl)-2-hydroxy-1,3-diaminopropane, 2,6-bis-(bis-(2-pyridylmethyl)aminomethyl)-4-methylphenol, 1,3-bis-(bis-(2-benzimidazolylmethyl)aminomethyl) benzene, sorbitol, mannitol, erythritol, adonitol, inositol, lactose, and optionally substituted salens, porphins and porphyrins. The inorganic neutral ligands include in particular ammonia and water. If not all coordination sites of the transition metal central atom are occupied by neutral ligands, the complex contains further, preferably anionic, ligands, of these in particular mono- or bidentate ligands. These include in particular the halides, such as fluoride, chloride, bromide and iodide, and the (NO₂)—group, i.e., a nitro ligand or a nitrito ligand. The (NO₂)⁻ group may also be bonded to a transition metal to form a chelate or it may asymmetrically bridge or μ1-O-bridge two transition metal atoms. In addition to the ligands mentioned, the transition metal complexes may carry further, generally more simple, ligands, in particular mono- or polyvalent anion ligands. For example, nitrate, acetate, trifluoroacetate, formate, carbonate, citrate, oxalate, perchlorate, and complex anions, such as hexafluorophosphate are suitable. The anion ligands are intended to ensure charge balance between the transition metal central atom and the ligand system. The presence of oxo ligands, peroxo ligands and imino ligands is also possible. In particular, such ligands can also have a bridging effect, such that polynuclear complexes are produced. In the case of bridged, binuclear complexes, the two metal atoms in the complex do not need to be the same. The use of binuclear complexes in which the two transition metal central atoms have different oxidation numbers is also possible. If anion ligands are missing or the presence of anionic ligands does not result in charge balance in the complex, anionic counterions which neutralize the cationic transition metal complex are present in the transition metal complex compounds to be used according to the invention. These anionic counterions include in particular nitrate, hydroxide, hexafluorophosphate, sulfate, chlorate, perchlorate, the halides, such as chloride, or the anions of carboxylic acids, such as formate, acetate, oxalate, benzoate or citrate. Examples of transition metal complex compounds that can be used are [N,N′-bis[(2-hydroxy-5-vinylphenyl)methylene]-1,2-diamino-cyclohexane]manganese(III) chloride, [N,N′-bis[(2-hydroxy-5-nitrophenyl)methylene]-1,2-diamino-cyclohexane]manganese(III) acetate, [N,N′-bis[(2-hydroxyphenyl)methylene]-1,2-phenylenediamine]manganese(III) acetate, [N,N′-bis[(2-hydroxyphenyl)methylene]-1,2-diaminocyclohexane]manganese(III) chloride, [N,N′-bis[(2-hydroxyphenyl)methylene]-1,2-diaminoethane]manganese(III) chloride, [N,N′-bis[(2-hydroxy-5-sulfonato-phenyl)methylene]-1,2-diaminoethane]manganese(III) chloride, manganese oxalato complexes, nitropentamminecobalt(III) chloride, nitritopentaamminecobalt(III) chloride, hexamminecobalt(III) chloride, chloropentamminecobalt(III) chloride and the peroxo complex [(NH₃)₅Co—O—O—Co(NH₃)₅]Cl₄.

Enzymes from the class of proteases, amylases, lipases, cutinases, pullulanases, hemicellulases, cellulases, oxidases, laccases and peroxidases, and mixtures thereof are suitable as enzymes that can be used in washing and cleaning agents. Enzymatic active ingredients obtained from fungi or bacteria, such as Bacillus subtilis, Bacillus licheniformis, Bacillus lentus, Streptomyces griseus, Humicola lanuginosa, Humicola insolens, Pseudomonas pseudoalcaligenes, Pseudomonas cepacia or Coprinus cinereus are particularly suitable. The enzymes can be adsorbed on carrier substances and/or embedded in coating substances to protect the enzymes from premature inactivation. They are preferably present in the washing or cleaning agents according to the invention in amounts of up to 5 wt. %, in particular from 0.002 wt. % to 4 wt. %. If the agent according to the invention contains protease, it preferably has a proteolytic activity in the range of approximately 100 PE/g to approximately 10,000 PE/g, in particular 300 PE/g to 8,000 PE/g. If a plurality of enzymes is to be used in the agent according to the invention, this can be carried out by incorporation of the two or more separate enzymes or enzymes that have been separately manufactured in a known manner, or by two or more enzymes manufactured together in a granulate.

In order to set a desired pH that does not result automatically from mixing the other components, the agents according to the invention may contain acids that are compatible with the system and environment, in particular citric acid, acetic acid, tartaric acid, malic acid, lactic acid, amino acids, such as glycine, glutaminic acid, arginine and/or aspartic acid, glycolic acid, maleic acid, fumaric acid, salicylic acid, succinic acid, glutaric acid, and/or adipic acid, but optionally also mineral acids, in particular sulfuric acid, or bases, in particular ammonium or alkali hydroxides. pH regulators of this kind are present in washing and cleaning agents according to the invention in amounts of preferably no greater than 20 wt. %, in particular of 1.2 wt. % to 17 wt. %. Organic acids are preferably used in hair treatment agents. Lactic acid and/or maleic acid are particularly preferably used as organic acids. The organic acids can be used to adjust the pH value of the hair treatment agent, particularly to a value between 3.5 and 5. In a preferred embodiment, the hair treatment agent has a pH in the range of 3.75 to 4.75.

The object of graying inhibitors is to keep the dirt that is removed from textile fibers suspended in the liquor. Water-soluble colloids, which are usually organic, are suitable for this purpose, for example starch, sizing material, gelatin, salts of ethercarboxylic acids or ethersulfonic acids of starch or of cellulose, or salts of acidic sulfuric acid esters of cellulose or of starch. Water-soluble polyamides containing acid groups are also suitable for this purpose. Starch derivatives other than those mentioned above may also be used, for example aldehyde starches. Cellulose ethers, such as carboxymethyl cellulose (Na salt), methyl cellulose, hydroxyalkyl cellulose, and mixed ethers, such as methyl hydroxyethyl cellulose, methyl hydroxypropyl cellulose, methyl carboxymethyl cellulose and mixtures thereof, are preferably used, for example in amounts of 0.1 to 5 wt. %, based on the washing agent.

If desired, washing agents may contain a conventional dye transfer inhibitor, preferably then in amounts of up to 2 wt. %, in particular 0.1 wL % to 1 wt. %, which, in a preferred embodiment, is selected from the polymers of vinylpyrrolidone, vinylimidazole, vinylpyridine-N-oxide, or the copolymers thereof. Both polyvinylpyrrolidones having molecular weights of 15,000 g/mol to 50,000 g/mol and polyvinylpyrrolidones having higher molecular weights of, for example, up to more than 1,000,000 g/mol, in particular of 1,500,000 g/mol to 4,000,000 g/mol, N-vinylimidazole-N-vinylpyrrolidone copolymers, polyvinyloxazolidones, copolymers based on vinyl monomers and carboxylic acid amides, pyrrolidone radical-containing polyesters and polyamides, grafted polyamidoamines and polyethyleneimines, polyamine-N-oxide polymers and polyvinyl alcohols are usable. However, it is also possible to use enzymatic systems comprising a peroxidase and hydrogen peroxide or a substance which produces hydrogen peroxide in water. The addition of a mediator compound for the peroxidase, for example an acetosyringone, a phenol derivative or a phenotiazine or phenoxazine, is preferred in this case, it also being possible to additionally use above-mentioned polymeric dye transfer inhibitor active ingredients. Polyvinylpyrrolidone preferably has an average molar mass in the range of from 10,000 g/mol to 60,000 g/mol, in particular in the range of from 25,000 g/mol to 50,000 g/mol. Of the copolymers, those consisting of vinylpyrrolidone and vinylimidazole in a molar ratio of 5:1 to 1:1 with an average molar mass in the range of 5,000 g/mol to 50,000 g/mol, in particular 10,000 g/mol to 20,000 g/mol, are preferred. In preferred embodiments of the invention, the washing agents are, however, free of additional dye transfer inhibitors of this kind.

Washing agents may contain, for example, derivatives of diaminostilbene disulfonic acid or the alkali metal salts thereof as optical brighteners, although they are preferably free of optical brighteners when used as color washing agents. Salts of 4,4′-bis(2-anilino-4-morpholino-1,3,5-triazinyl-6-amino)stilbene-2,2′-disulfonic acid or compounds having a similar structure which, instead of the morpholino group, have a diethanolamino group, a methylamino group, an anilino group or a 2-methoxyethylamino group are suitable, for example. Furthermore, brighteners of the substituted diphenyl styryl type may be present, for example the alkali salts of 4,4′-bis(2-sulfostyryl)diphenyl, 4,4′-bis(4-chloro-3-sulfostyryl)diphenyl, or 4-(4-chlorostyryl)-4′-(2-sulfostyryl)diphenyl. Mixtures of the aforementioned optical brighteners may also be used.

It may be advantageous to add conventional foam inhibitors to the washing agents, in particular during use in mechanical processes. Soaps of natural or synthetic origin having a high proportion of C₁₈-C₂₄ fatty acids are suitable as foam inhibitors, for example. Suitable non-surfactant foam inhibitors are, for example, organopolysiloxanes and mixtures thereof with microfine, optionally silanated silicic acid and paraffins, waxes, microcrystalline waxes, and mixtures thereof with silanated silicic acid or bis fatty acid alkylenediamides. Mixtures of various foam inhibitors are also advantageously used, for example those consisting of silicones, paraffins, or waxes. The foam inhibitors, in particular silicone and/or paraffin-containing foam inhibitors, are preferably bonded to a granular carrier substance that is soluble or dispersible in water. Mixtures of paraffins and bistearylethylenediamide are particularly preferred.

In a preferred embodiment, a washing or cleaning agent according to the invention is in particulate form and contains, in addition to the surfactant that is essential to the invention, builders, in particular in an amount in the range of 1 wt. % to 60 wt. %.

In a further preferred embodiment, a washing or cleaning agent according to the invention is liquid and contains up to 90 wt. %, in particular 10 wt. % to 85 wt. %, preferably 25 wt. % to 75 wt. %, and particularly preferably 35 wt. % to 65 wt. %, water, water-miscible solvent, or a mixture of water and water-miscible solvent. Water-miscible solvents include, for example, monohydric alcohols having 1 to 4 C atoms, in particular methanol, ethanol, isopropanol and tert-butanol, diols and triols having 2 to 4 C atoms, in particular ethylene glycol, propylene glycol and glycerol, and mixtures thereof, and the ethers that can be derived from the classes of compounds mentioned. Water-miscible solvents of this kind are present in the washing or cleaning agents according to the invention preferably in amounts of no greater than 30 wt. %, in particular from 2 wt. % to 20 wt. %. A liquid washing or cleaning agent preferably has a pH of at least pH 5.6, in particular in the range of pH 7 to pH 9.

A hair treatment agent according to the invention is preferably liquid and contains an aqueous or an aqueous-alcoholic carrier. An aqueous carrier contains at least 50 wt. % water. Aqueous-alcoholic carriers are understood to mean aqueous solutions containing 3 to 70 wt. % of a C₂-C₆ alcohol, in particular ethanol or propanol, isopropanol, butanol, isobutanol, tert-butanol, n-pentanol, iso-pentanols, n-hexanol, iso-hexanols, glycol, glycerin, 1,2-pentanediol, 1,5-pentanediol, 1,2-hexanediol or 1,6-hexanediol. The agents can additionally contain further organic solvents, such as for example methoxybutanol, benzyl alcohol, ethyl diglycol or 1,2-propylene glycol. Thus all water-soluble organic solvents are preferred.

In a further preferred embodiment, an agent according to the invention is portioned ready for individual metering in a chamber made of water-soluble material; the agent then preferably contains less than 15 wt. %, in particular in the range of 1 wt. % to 12 wt. %, water. A portion is an independent dosing unit having at least one chamber in which the product to be dosed is contained. A chamber is a space delimited by walls (e.g. by a film), which space can also exist without the product to be dosed (optionally by changing its shape). A surface coating or a layer of a surface coating is therefore not a wall according to the present invention.

The walls of the chamber are made of a water-soluble material. The water solubility of the material can be determined by means of a square film of said material (film: 22×22 mm with a thickness of 76 μm) fixed in a square frame (edge length on the inside: 20 mm) according to the following measurement protocol. Said framed film is submerged into 800 ml of distilled water, temperature-controlled to 20° C., in a 1 liter glass beaker with a circular base (Schott, Mainz, 1000 ml glass beaker, low shape), so that the surface of the tensioned film is arranged at a right angle to the base of the beaker, the upper edge of the frame is 1 cm below the water surface, and the lower edge of the frame is oriented parallel to the base of the glass beaker such that the lower edge of the frame extends along the radius of the base of the glass beaker and the center of the lower edge of the frame is arranged above the center of the radius of the glass beaker bottom. The material dissolves within 600 seconds when stirred (stirring speed magnet stirrer 300 rpm, stirring rod: 5 cm long), such that no solid particles at all can be seen with the naked eye.

The walls of the chambers and therefore the water-soluble wrappings of the washing agents according to the invention are preferably formed by a water-soluble film material. Water-soluble packages of this kind can be produced either by means of methods of vertical form fill sealing or by means of thermoforming methods.

The thermoforming method generally includes forming a first layer from a water-soluble film material in order to produce bulges for receiving a composition, pouring the composition into the bulges, covering the bulges filled with the composition with a second layer made of a water-soluble film material, and sealing the first and second layers to one another at least around the bulges.

The water-soluble film material is preferably selected from polymers or polymer mixtures. The wrapping may be made up of one or of two or more layers of water-soluble film material. The water-soluble film materials of the first layer and of the additional layers, if present, may be the same or different.

It is preferable for the water-soluble wrapping to contain polyvinyl alcohol or a polyvinyl alcohol copolymer; particularly preferably, it consists of polyvinyl alcohol or polyvinyl alcohol copolymer.

Water-soluble films for producing the water-soluble wrapping are preferably based on a polyvinyl alcohol or a polyvinyl alcohol copolymer of which the molecular weight is in the range of 10,000 to 1,000,000 gmol⁻¹, preferably of 20,000 to 500,000 gmol⁻¹, particularly preferably of 30,000 to 100,000 gmol⁻¹, and in particular of 40,000 to 80,000 gmol⁻¹.

Polyvinyl alcohol is usually prepared by hydrolysis of polyvinyl acetate, since the direct synthesis route is not possible. The same applies to polyvinyl alcohol copolymers, which are prepared accordingly from polyvinyl acetate copolymers. It is preferable for at least one layer of the water-soluble wrapping to include a polyvinyl alcohol of which the degree of hydrolysis is 70 to 100 mol. %, preferably 80 to 90 mol. %, particularly preferably 81 to 89 mol. %, and in particular 82 to 88 mol. %.

Polymers selected from the group comprising acrylic acid-containing polymers, polyacrylamides, oxazoline polymers, polystyrene sulfonates, polyurethanes, polyesters, polyethers, polylactic acid, and/or mixtures of the above polymers may additionally be added to a film material suitable for producing the water-soluble wrapping. It is also possible to copolymerize such monomers on which the polymers are based, individually or in mixtures of two or more, with vinyl acetate.

Preferred polyvinyl alcohol copolymers comprise, in addition to vinyl alcohol, an ethylenically unsaturated carboxylic acid, or the salt or ester thereof. Polyvinyl alcohol copolymers of this kind particularly preferably contain acrylic acid, methacrylic acid, acrylic acid ester, methacrylic acid ester or mixtures thereof in addition to vinyl alcohol; of the esters, C₁₋₄ alkyl esters or C₁₋₄ hydroxyalkyl esters are preferred. Polyvinyl alcohol copolymers which include, in addition to vinyl alcohol, ethylenically unsaturated dicarboxylic acids as further monomers are also preferred. Suitable dicarboxylic acids are, for example, itaconic acid, maleic acid, fumaric acid, and mixtures thereof, itaconic acid being particularly preferred.

Suitable water-soluble films for use in the wrappings of the water-soluble packaging according to the invention are films which are sold by MonoSol LLC, for example under the names M8630, C8400 or M8900. Other suitable films include films with the name Solublon® PT, Solublon® GA, Solublon® KC or Solublon® KL by Aicello Chemical Europe GmbH or the VF—HP films by Kuraray.

The washing or cleaning agent portion comprising the washing or cleaning agent and the water-soluble wrapping may have one or more chambers. The water-soluble wrappings having a chamber can have a substantially dimensionally stable spherical, rotationally ellipsoidal, cubic, cuboidal or pillow-shaped design with a circular, elliptical, square or rectangular basic shape. The agent may be contained in one or more chambers, if present, of the water-soluble wrapping.

In a preferred embodiment, the water-soluble wrapping has two chambers. In this embodiment, the two chambers may each contain a solid partial composition or a liquid partial composition, or the first chamber contains a liquid partial composition and the second chamber a solid partial composition.

The proportions of the agents contained in the different chambers of a water-soluble wrapping having two or more chambers may have the same composition. Preferably, however, the agents in a water-soluble wrapping having at least two chambers have partial compositions which differ by at least one ingredient and/or by the content of at least one ingredient. Preferably, a partial composition of such agents according to the invention comprises an enzyme and/or a bleach activator and a separate further partial composition comprises peroxidic bleaching agent, the first partial composition thus in particular not comprising peroxidic bleaching agent and the second partial composition in particular not comprising an enzyme or a bleach activator.

Packaging portions in a water-soluble wrapping allows the user, for one application, to put one or, if desired, a plurality, preferably one, of the portions into the washing machine or dishwasher, in particular into the detergent drawer of a washing machine, or into a container for carrying out a manual washing or cleaning method. Portion packs of this kind fulfil the consumer's desire for simplified dosage. After the addition of water, the wrapping material dissolves such that the ingredients can be released and take effect in the liquor. Preferably, a water-solubly wrapped portion weighs 10 g to 35 g, in particular 12 g to 28 g, and particularly preferably 12 g to 15 g, where 0.3 g to 2.5 g, in particular 0.7 g to 1.2 g, of the content of the water-soluble wrapping included in the stated weight is omitted.

The preparation of solid agents according to the invention presents no difficulties and can be carried out in a known manner, for example by spray-drying or granulation, with enzymes and any other heat-sensitive ingredients, such as bleaching agents optionally being added separately later on. For the preparation of agents having an increased bulk weight, in particular in the range of from 650 g/l to 950 g/l, a method having an extrusion step is preferred.

Liquid or pasty agents according to the invention in the form of solutions typically containing water solvents are usually prepared by simple mixing of the ingredients, which can be put into an automatic mixer in bulk or as a solution.

### EXAMPLES

### Example 1: Washing Performance

In a high throughput washing device, the washing performance of a sophorolipid (Rewoferm® SL ONE) in combination with a cationic surfactant (C_{12/14}-dimethyl-hydroxyethyl-ammonium chloride (Präpagen® HY) or benzalkonium chloride) each time was investigated at 30° C. and pH 8. For comparison, the commercially available sophorolipid and the pure acid form of the sophorolipid (i.e. the hydrolyzed Rewoferm® SL ONE) were also examined on their own. Different molar ratios of the sophorolipid acid form to the cation surfactant were investigated (given in Tables 1 and 2). The total surfactant concentration in all tests was 0.62 g/l in the washing liquor. 12 different standardized types of soiling (3*lipstick, pigment/oil, pigment/sebum, olive oil, 2*sebum, mayonnaise, frying fat, lard, beef dripping) were used on polyester-cotton blends. After drying, the reflectance values of the washed textiles were determined and added. In the following tables, the results are given as mean values of triplicate determinations of the 12 different types of soiling:

TABLE 1
SUM OF REFLECTANCE VALUES

| | |
|---|---|
| Sophorolipid | 600.4 |
| Sophorolipid acid form | 593.9 |
| Sophorolipid acid form/Präpagen ® HY 1:1 | 630.6 |
| Sophorolipid acid form/Präpagen ® HY 1:0.9 | 644.9 |
| Sophorolipid acid form/Präpagen ® HY 1:0.7 | 649.5 |
| Sophorolipid acid form/Präpagen ® HY 1:0.5 | 646.5 |
| Sophorolipid acid form/Präpagen ® HY 1:0.3 | 640.6 |
| Sophorolipid acid form/Präpagen ® HY 1:0.1 | 613.4 |

TABLE 2
SUM OF REFLECTANCE VALUES

| | |
|---|---|
| Sophorolipid | 597.7 |
| Sophorolipid acid form | 591.1 |
| Sophorolipid acid form/Präpagen ® HY 2:1 | 634.0 |
| Sophorolipid acid form/Benzalkonium chloride 2:1 | 613.4 |
| Sophorolipid/benzalkonium chloride 2:1 | 594.9 |

While the sophorolipid showed a low washing performance both in the commercial form and in the pure acid form, the latter was significantly improved by being combined with a cationic surfactant.

### Example 2: Foam Formation

In 50 ml of a solution containing one of the surfactants specified in Table 2 in a concentration of 0.12 g/l, foam was created in a TECLIS Foam Scan® device by constant gassing through a glass frit from below for 120 seconds with an air flow of 100 ml/min and the foam heights were detected by a camera. Rewoferm® SL ONE, also used in Example 1, was used as the sophorolipid.

TABLE 3
FOAM VOLUME [ML] AFTER 120 SECONDS

| | |
|---|---|
| Sophorolipid acid form | 46.0 |
| Präpagen ® HY 1:1 | 118.0 |
| Sophorolipid acid form/Präpagen ® HY | 1:1215.6 |

1. A compound of general formula (I),
S—CO₂⁻N⁺(R¹R²R³R⁴) (I)
where
S—CO₂⁻ is a sophorolipid carboxylate anion and
R¹ is selected from R² and H, R² is selected from C₁ to C₄ alkyl radicals and C₂ to C₄ hydroxyalkyl radicals, R³ is selected from R⁴ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, wherein R⁵ is selected from the C₂ to C₄ alkylene radicals and R⁶ is selected from the C₁₀ to C₁₈ alkyl radicals, and in each of R¹, R², R³, R⁴ and R⁶, the alkyl moieties can be linear or branched-chain.

2. A mixture of compounds of general formula (I),
S—CO₂⁻N⁺(R¹R²R³R⁴) (I)
where
S—CO₂⁻ is a sophorolipid carboxylate anion and
R¹ is selected from R² and H, R² is selected from C₁ to C₄ alkyl radicals and C₂ to C₄ hydroxyalkyl radicals, R³ is selected from R⁴ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, wherein R⁵ is selected from the C₂ to C₄ alkylene radicals and R⁶ is selected from the C₁₀ to C₁₈ alkyl radicals, and in each of R¹, R², R³, R⁴ and R⁶, the alkyl moieties can be linear or branched-chain with sophorolipids which do not have an ammonium countercation as defined in formula (I), but are present in the form of the free acid or its alkali salts, or which are present in the lactone form.

3. A mixture of compounds of general formula (I),
S—CO₂⁻N⁺(R¹R²R³R⁴) (I)
where
S—CO₂⁻ is a sophorolipid carboxylate anion and
R¹ is selected from R² and H, R² is selected from C₁ to C₄ alkyl radicals and C₂ to C₄ hydroxyalkyl radicals, R³ is selected from R⁴ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, wherein R⁵ is selected from the C₂ to C₄ alkylene radicals and R⁶ is selected from the C₁₀ to C₁₈ alkyl radicals, and in each of R¹, R², R³, R⁴ and R⁶, the alkyl moieties can be linear or branched-chain with salts containing cations which correspond to N⁺(R¹R²R³R⁴) in formula (I) but have a different counteranion.

4. The mixture according to claim 2, characterized in that said mixture contains a) the compound of general formula (I) and b) the sophorolipid not defined in general formula (I) and/or the cation not defined in general formula (I) in weight ratios of a) to b) in ranges from 1:100 to 100:1.

5. A washing, cleaning or hair-treatment agent containing a compound according to general formula (I),
S—CO₂⁻N⁺(R¹R²R³R⁴) (I)
where
S—CO₂⁻ is a sophorolipid carboxylate anion and
R¹ is selected from R² and H, R² is selected from C₁ to C₄ alkyl radicals and C₂ to C₄ hydroxyalkyl radicals, R³ is selected from R⁴ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, wherein R⁵ is selected from the C₂ to C₄ alkylene radicals and R⁶ is selected from the C₁₀ to C₁₈ alkyl radicals, and in each of R¹, R², R³, R⁴ and R⁶, the alkyl moieties can be linear or branched-chain, or a mixture according to claim 2.

6. The agent according to claim 5, characterized in that said agent contains 0.1 wt. % to 40 wt. % of the compound of general formula (I) or the mixture.

7. A method for washing textiles, during which method a compound of general formula (I),
S—CO₂—N⁺(R¹R²R³R⁴) (I)
where
S—CO₂⁻ is a sophorolipid carboxylate anion and
R¹ is selected from R² and H, R² is selected from C₁ to C₄ alkyl radicals and C₂ to C₄ hydroxyalkyl radicals, R³ is selected from R⁴ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, wherein R⁵ is selected from the C₂ to C₄ alkylene radicals and R⁶ is selected from the C₁₀ to C₁₈ alkyl radicals, and in each of R¹, R², R³, R⁴ and R⁶, the alkyl moieties can be linear or branched-chain, or a mixture according to claim 2, or a washing agent containing a compound of general formula (I), or a washing agent containing a mixture according to claim 2, is introduced into a washing liquor which contains a textile in need of cleaning or into which a textile in need of cleaning is introduced.

8. A method for cleaning hard surfaces, during which method a compound of general formula (I),
S—CO₂⁻N⁺(R¹R²R³R⁴) (I)
where
S—CO₂⁻ is a sophorolipid carboxylate anion and
R¹ is selected from R² and H, R² is selected from C₁ to C₄ alkyl radicals and C₂ to C₄ hydroxyalkyl radicals, R³ is selected from R⁴ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, wherein R⁵ is selected from the C₂ to C₄ alkylene radicals and R⁶ is selected from the C₁₀ to C₁₈ alkyl radicals, and in each of R¹, R², R³, R⁴ and R⁶, the alkyl moieties can be linear or branched-chain, or a mixture according to claim 2, or a cleaning agent containing a compound of general formula (I), or a cleaning agent containing a mixture according to claim 2, is introduced into a cleaning liquor which is brought into contact with the hard surface to be cleaned.

9. A method of treating hair, during which method a compound of general formula (I),
S—CO₂⁻N⁺(R¹R²R³R⁴) (I)
where
S—CO₂⁻ is a sophorolipid carboxylate anion and
R¹ is selected from R² and H, R² is selected from C₁ to C₄ alkyl radicals and C₂ to C₄ hydroxyalkyl radicals, R³ is selected from R⁴ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, wherein R⁵ is selected from the C₂ to C₄ alkylene radicals and R⁶ is selected from the C₁₀ to C₁₈ alkyl radicals, and in each of R¹, R², R³, R⁴ and R⁶, the alkyl moieties can be linear or branched-chain, or a mixture according to claim 2, or a hair treatment agent containing a compound of general formula (I), or a hair treatment agent containing a mixture according to claim 2, is applied in particular to wet hair.

10. The compound according to claim 1, characterized in that in the cations N⁺(R¹R²R³R⁴) of the compounds of general formula (I), two to three of the four radicals have a carbon chain length of at most and the remaining radical or the other two radicals have a carbon chain of C₁₀ to C₁₈.

11. The mixture according to claim 3, characterized in that said mixture contains a) the compound of general formula (I) and b) the sophorolipid not defined in general formula (I) and/or the cation not defined in general formula (I) in weight ratios of a) to b) in ranges from 1:100 to 100:1.

12. A washing, cleaning or hair-treatment agent containing a compound according to general formula (I),
S—CO₂⁻N⁺(R¹R²R³R⁴) (I)
where
S—CO₂⁻ is a sophorolipid carboxylate anion and
R¹ is selected from R² and H, R² is selected from C₁ to C₄ alkyl radicals and C₂ to C₄ hydroxyalkyl radicals, R³ is selected from R⁴ and R², and R⁴ is selected from C₁₀ to C₁₈ alkyl radicals, C₇ to C₂₀ alkylaryl radicals, —R⁵—O(C═O)—R⁶ radicals and —R⁵—NH(C═O)—R⁶ radicals, wherein R⁵ is selected from the C₂ to C₄ alkylene radicals and R⁶ is selected from the C₁₀ to C₁₈ alkyl radicals, and in each of R¹, R², R³, R⁴ and R⁶, the alkyl moieties can be linear or branched-chain, or a mixture according to claim 3.

13. The agent according to claim 12, characterized in that said agent contains 0.1 wt. % to 40 wt. % of the compound of general formula (I) or the mixture.

14. The mixture according to claim 2, characterized in that in the cations N⁺(R¹R²R³R⁴) of the compounds of general formula (I), two to three of the four radicals have a carbon chain length of at most 4 and the remaining radical or the other two radicals have a carbon chain of C₁₀ to C₁₈.

15. The mixture according to claim 3, characterized in that in the cations N⁺(R¹R²R³R⁴) of the compounds of general formula (I), two to three of the four radicals have a carbon chain length of at most 4 and the remaining radical or the other two radicals have a carbon chain of C₁₀ to C₁₈.

16. The agent according to claim 5, characterized in that in the cations N⁺(R¹R²R³R⁴) of the compounds of general formula (I), two to three of the four radicals have a carbon chain length of at most 4 and the remaining radical or the other two radicals have a carbon chain of C₁₀ to C₁₈.

17. The method according to claim 8, characterized in that in the cations N⁺(R¹R²R³R⁴) of the compounds of general formula (I), two to three of the four radicals have a carbon chain length of at most 4 and the remaining radical or the other two radicals have a carbon chain of C₁₀ to C₁₈.
